(19) United States
(12) Patent Application Publication
(10) Pub. No.: US 2024/0358484 A1
(43) Pub. Date: Oct. 31, 2024

(54) MULTICOLOR ZIRCONIUM OXIDE BLANK FOR DENTAL PROSTHESES

(71) Applicant: Ivoclar Vivadent AG, Schaan (LI)

(72) Inventors: Jose PULIDO, Widnau (CH); Frank ROTHBRUST, Röns (AT); Corina SERBAN, Lindau (DE); Sebastian KROLIKOWSKI, Lachen (CH); Roman CADUFF, Paspels (CH); Christian RITZBERGER, Grabs (CH)

(21) Appl. No.: 18/682,654

(22) PCT Filed: Aug. 10, 2022
(86) PCT No.: PCT/EP2022/072435

§ 371 Date: Feb. 9, 2024

(30) Foreign Application Priority Data
Aug. 10, 2021 (EP) 21190668.0

(51) Int. Cl. A61C 13/083; A61C 13/00

(52) U.S. Cl. CPC A61C 13/083; A61C 13/0006; A61C 13/0022

(57) ABSTRACT

The present invention relates to a blank for dental prostheses which has a first layer based on zirconium oxide ceramic and a second layer based on zirconium oxide ceramic, the first layer and the second layer differing in color and forming a boundary surface, wherein the boundary surface is formed in the course of the dental arch in an undulating shape with alternating wave troughs and wave crests, and the vertex lines of the wave crests, viewed in plan view of the boundary surface, extend radially in the mesial-distal direction.

The invention relates to a blank for dental purposes, in particular for the manufacture of dental restorations such as dental prostheses, with which the optical properties of natural tooth material and the natural oral mucosa can be imitated very well and which, due to its properties, is particularly suitable for the simple manufacture of aesthetically sophisticated dental prostheses, such as maxillary full dentures and/or mandibular full dentures, with very good mechanical properties.

In the dental field, multi-unit and to some extent wide-span implant-supported restorations are increasingly being fabricated nowadays for the treatment of patients in the lower and upper jaw.

In addition to the aesthetics of the teeth, the color matching of the transition to the oral mucosa (gingiva) and the optimal color imitation of the actual oral mucosa play an essential role for the overall aesthetic result. However, a high manual effort is required to restore the gingival area in a lifelike manner.

In the past, full dentures were usually fabricated by taking a silicone impression of the patient's edentulous jaw; fabricating a plaster model on this basis; setting up an artificial row of teeth on the plaster model with the aid of a wax plate; carrying out a try-in on the patient and, if necessary, a post-correction; embedding the artificial teeth, the wax model and part of the plaster model in plaster; melting out the wax; filling the resulting cavity with a PMMA-based denture material; removing the plaster embedding; and carrying out a final cleaning and surface polishing. This procedure usually produces very good results, but is very time-consuming and labor-intensive.

Multi-colored blocks simulating the gradient of color and translucency from dentin to enamel and their use in dental technology are known from the prior art. Blanks based on zirconium oxide ceramics and their use for the manufacture of dental prostheses in a CAD/CAM process by means of a computer-controlled milling machine are also already known from the prior art. However, even when using such blanks, a high degree of manual effort is required to restore the gingival area in a lifelike manner. This is mainly due to the fact that, according to a first alternative, the gingival area has to be applied subsequently, e.g. by layering or pressing the gingiva portion onto a ZrO₂ restoration by means of gingiva-colored press ingots and recreating it true-to-nature by means of all-ceramic techniques. It is also possible to layer the gingival area with composite material after completion of the restoration. However, there is a risk of color instability and plaque or odor formation.

According to a second alternative, a zirconium oxide blank with a pre-shaped gingiva area is used, which, however, still requires post-treatment in order to achieve an optimal color imitation of the gingiva. When using tooth-colored, pre-shaded zirconium oxide discs with a pre-shaped gingival area, the tooth-colored area is covered with glass-ceramic layering, veneering, coloring and characterization materials in several steps. When using non-colored or slightly colored zirconium oxide discs with a preformed gingival portion, the problem is currently solved by infiltrating a highly concentrated solution based on water-soluble Er³⁺ salts, such as ErCl₃, ErCl₃.6H₂O or Er(NO₃)₃.5H₂O, in the gingival area.

The highly concentrated infiltration solutions based on Er³⁺ have a strongly acidic pH value, often in the range of 1-3. For safety reasons, there is therefore a very high risk of the user being burned or inhaling harmful gases/vapors, such as HCl or HNO₃ vapors, if used improperly when opening the containers without using a suitable fume cupboard or adequate room ventilation. Furthermore, such solutions are usually not stable in the long term and therefore change their properties over time. Uncontrolled evaporation of the solvent can also lead to changes in concentration over time. Uncontrolled complex formation can also occur, as these solutions usually also contain certain organic compounds. In the worst case, this leads to the precipitation of certain compounds.

Infiltration with coloring solution may further result in the coloring solution not being homogeneously distributed in the porous zirconium oxide. This may depend on the user as well as on the surface quality of the restoration, such as the degree of dust-freeness and the moisture content. Inhomogeneities can have a significant impact on the final result.

Infiltration in the gingival area also causes a change in the degree of stabilization and the phase composition of the zirconium oxide after the final sintering. During the final sintering, Er³⁺ ions can be incorporated into the crystal framework in addition to the Y³⁺ ions already incorporated into the zirconium oxide. This then leads to over-stabilization and an associated change in phase composition in the infiltrated areas. This results in a deterioration of mechanical properties, especially fracture toughness and biaxial strength. Especially in the gingiva area, a very large chewing load is absorbed by implant-supported restorations, so that a weakening of this area must be avoided.

Furthermore, doping with Er₂O₃ in higher concentrations changes the sintering behavior of the zirconium oxide. Er₂O₃ acts as a sintering activator and changes the theoretical density of the stabilized zirconium oxide. The total shrinkage of the infiltrated area increases so that the enlargement factor taken into account when milling the ingot is no longer correct and the accuracy of fit of the densely sintered restoration is negatively affected. If Er₂O₃ is only used locally to color the gingival area, local shrinkage occurs earlier during sintering, i.e. while the gingival area is already shrinking, the restoration in the tooth area lags behind the shrinkage. This leads to stresses during the sintering process that remain in the overall construction and weaken it permanently. Sudden fractures often occur at the workplace or when the restoration is placed.

WO 2010/057584 A1 proposes a milling block with a pink-colored component and a tooth-colored component to simplify the fabrication of dentures, with both the pink-colored component and the tooth-colored component being essentially made of PMMA or a (meth) acrylate-based plastic. Furthermore, the use of such a milling block for the fabrication of a maxillary full denture or a mandibular full denture with a CAD/CAM process by means of a numerically controlled milling unit is described.

WO 2013/068124 also describes a milling block with two areas for the production of dental prostheses. The first area has a denture base that can be machined corresponding to the shape of the jaw and adapted to the patient's individual mucosa by milling. The second area has non-individualized, prefabricated artificial teeth that are set up in a predetermined arrangement and do not require any further finishing. Among other materials, zirconium oxide ceramics are mentioned as the material for the first and second area. However, an adaptation to the individual patient situation in the opposite jaw is not possible with such a milling block.

US 2021/0128283 A1 describes a two-colored blank for the fabrication of a prosthesis by means of CAD/CAM, with the blank having a lower pink-colored layer for imitating the gingiva and an upper tooth-colored layer for imitating teeth. Among other things, zirconium oxide ceramics are mentioned as the material for the upper and lower layers. The boundary surface between the two layers has a plurality of convex portions and concave portions, so that the upper surface of the gingival layer has an undulating shape. It is described that in this way the natural transition to the gingiva can be imitated particularly easily and it is possible to reduce the number of shade correction steps.

EP 3 064 170 A1 and EP 3 597 143 A1 each disclose a denture blank composed of a flesh-colored resin material and a tooth-colored resin material, wherein the boundary surface between the materials, as viewed in the dental arch direction, is undulated.

It is therefore a great challenge to provide blanks that meet the diverse requirements for use in the field of dental technology, especially for the production of dental prostheses. Such blanks should not only be easy to manufacture, but they should also be easy to shape into the desired geometry and still produce highstrength restorations. Finally, the blanks should result in an appearance that is close to that of the natural tooth material and the natural oral mucosa, so that a costly subsequent creation of the desired optical properties of the dental prostheses can be omitted.

According to the invention, the above-mentioned problems are to be avoided. The invention is therefore based in particular on the problem of providing a blank which is easy to manufacture, to which the shape of the desired dental restoration can be given by machining in a simple manner and which, after shaping, can be converted into a precise and high-strength dental prosthesis, with the blank being able to very well imitate the optical appearance of natural tooth material and natural oral mucosa.

This problem is solved by the blank according to claims 1 to 18. It is also an object of the invention to provide the process for preparing the blank according to claims 19 to 21, the use of the blank according to claim 22, and the process for preparing dental prostheses according to claims 23 to 25.

The invention relates to a dental prosthesis blank, i.e. a dental prosthesis blank. The blank according to the invention is characterized in that it has a first layer based on zirconium oxide ceramic and a second layer based on zirconium oxide ceramic, the first layer and the second layer differing in color and forming a boundary surface, wherein the boundary surface is formed, in the course of the dental arch, in an undulating shape with alternating wave troughs and wave crests, and the vertex lines of the wave crests, viewed in plan view of the boundary surface, extends radially in the mesial-distal direction.

The term “based on” means that the first and the second layer of the blank comprise predominantly zirconium oxide, i.e. ZrO₂, based on the mass of the sum of all components of the layer. In addition to ZrO₂ and small amounts of the impurity HfO₂, the first and/or the second layer may comprise, for example, components for adjusting the color, such as Fe, Tb, Ce, Pr, Mn, Cr, Ni, Co, Nd, Dy, Eu, Er, V and/or Ti, and/or components for adjusting the sintering kinetics, such as Mg, Al, Y, Ce, La, Yb, Gd, Ga and/or In. Furthermore, the first and/or the second layer may contain mixtures of zirconium oxide with other ceramics or spinels in the form of a composite and/or mixtures of zirconium oxide with pigments.

Differences in color refer to differences in color shade in the narrower sense and/or differences in translucency, opalescence or fluorescence. The term “translucency” describes the light transmission. The color can be characterized in particular by its Lab value or by a shade guide commonly used in the dental industry. Furthermore, it is not necessary that the differences in the color of the first and second layers in the blank are visible to the human eye. Rather, a difference in shade and/or translucency may only become visible after a sintering step or heat treatment. Similarly, the term “color gradient” includes gradients of translucency, opalescence or fluorescence in addition to a gradient of hue.

The boundary surface between the first and second layer is formed in an undulating shape in the course of the dental arch of the dental prosthesis to be fabricated. This means that in a side view of an arc-shaped, in particular parabolic or semicircular, cut surface through the blank, the boundary surface between the first and second layer is wave-shaped. For example, if the blank has the shape of a disc, the cut surface extends from the upper base surface to the lower base surface of the disc and is substantially parallel to the lateral surface of the disc. The wave shape has alternating wave troughs and wave crests. The wave troughs can also be referred to as grooves or depressions and the wave crests can also be referred to as ridges or elevations. The peaks of a wave crest and also the-downward facing-peaks of a wave trough each form a vertex line. In other words, the vertexes of a wave crest or a wave trough each form a line, especially a straight line. The vertex lines of the wave crests, and preferably also the vertex lines of the wave troughs, extend from the inside to the outside, i.e. from mesial to distal, when viewed from above the boundary surface or the base surface of the blank. The term “undulating” is not used in the present context to describe only purely sinusoidal waveforms, but generally includes all waveforms with alternating raised and recessed areas.

Furthermore, the geometry of the boundary surface between the first and second layer of the blank is designed in such a way that the boundary surface is radially shaped in the mesial-distal direction. That is, in a top view of the boundary surface of the blank, the vertex lines of the wave crests extend from a central region of the blank in a radial direction, i.e. outwardly, in a ray shape, preferably in the form of straight lines. The three-dimensional undulating and radial geometry is preferably based on data from a large number of real patient cases.

The blank according to the invention is thus characterized in particular by the fact that a desired color gradient is created with the two differently colored layers as well as the integrated undulating and radial geometry of the layers, so that the color of teeth and the gingiva as well as the course of the transition from tooth material to gingiva can be imitated particularly well in the finished dental prosthesis. The undulating shape of the first layer can reproduce the gingival margin particularly well. Due to the radially extending wave structure, the gingival margin can always be created quasi automatically, regardless of the size of the required dental arch. This represents a particular advantage over the checkerboard-like distribution of convex and concave areas known from US 2021/0128283 A1.

Furthermore, the blank according to the invention is characterized by the fact that by using layers based on zirconium oxide ceramics, high-strength dental restorations can be produced that fully meet the requirements for the mechanical properties of e.g. long-span dental restorations, such as prostheses. In addition, the use of ZO as a material leads to further advantages. For example, in the case of zirconium oxide ceramics, the blank can be made flatter overall compared to blanks based on plastic materials, so that the overall height of the blank can be reduced and the machining of the blank in usual CAM milling units can be simplified, e.g. due to fewer undercuts in the design.

With the blank according to the invention, not only the optical appearance of natural tooth material and natural oral mucosa can be imitated very well. Furthermore, the blank according to the invention can be given the shape of the desired dental prosthesis in a particularly simple manner. Surprisingly, this is achieved by a combination of the special design of the boundary surface between the first and second layers and the use of zirconium oxide as the material of the first and second layers. The blank according to the invention enables efficient monolithic fabrication in the fields of digital fixed and conditionally removable prosthetics, so that a partial or total prosthesis can be fabricated in one milling process and few manual steps. Also, with the blank according to the invention, after shaping, e.g. by CAM milling, no subsequent infiltration with a coloring solution in the gingival area is necessary, which in the past has led to frequent cases of failure of the restoration in vivo. This guarantees a stress-free monolithic fabrication and thus a long-term stable restoration.

In a preferred embodiment of the blank according to the invention, the geometry of the boundary surface between the first and second layer of the blank is designed in such a way that the boundary surface, viewed in a top view of the boundary surface, is fan-shaped in the mesial-distal direction in the region of the anterior teeth of the prosthesis to be fabricated. That is, in a top view of the boundary surface of the blank, the vertex lines of the wave crests extend from a central point of a partial section of the dental arch to be created in a radial direction, i.e. outwards, in a fan shape. This means that in the area of the anterior teeth to be created, the wave crests, and preferably also wave troughs, extend in a fan shape when viewed in the oral-vestibular direction, i.e. radially starting from a ray-center.

Furthermore, it is preferred that the boundary surface between the first and second layer in the area of the molars to be created—viewed in the top view of the boundary surface—has radiating vertex lines of the wave troughs in the oral-buccal direction and, in particular, vertex lines of the wave troughs, and preferably also wave crests, that are essentially parallel to each other. The expression “essentially parallel” means that the vertex lines of the wave troughs, and preferably also wave crests, extend parallel or deviate from parallelism by at most 10 degrees, in particular at most 5 degrees. The design of parallel vertex lines of the wave troughs in the area of the molars to be created offers the advantage of better aesthetics in the posterior region in particular.

Overall, the two above embodiments of a fan-shaped design of the vertex lines of the wave crests in the anterior region and a parallel design of the vertex lines of the wave troughs in the posterior region allow to improve both the function and the aesthetics of the final dental prosthesis. In particular, it is possible to provide suitable tooth sets for both small and large dental arches. For large dental arches, the dental arch is milled slightly further radially outwards, i.e. displaced in the vestibular direction, and for small dental arches, it is milled further inwards, i.e. displaced orally. Due to the parallelization of the wave crests and wave troughs of the boundary surface for the area of the molars according to the invention, a comparatively large occlusal surface is also available for small dental arches according to the invention.

Thus, in a preferred embodiment, the blank according to the invention can have fan-shaped wave troughs and wave crests with a true ray-center in the area of the central incisors to be created. However, this is no longer properly hit by the vertex line of the wave crest for the canine to be created. Instead, this vertex line is less fan-shaped and more parallel to the adjacent vertex line of the lateral incisor. This also applies to the first premolar vertex line, which again has a course that is even closer to parallelism. The next vertex lines, i.e. the vertex lines for the second premolars, the first molars and the second molars, are completely parallel to each other.

Furthermore, the vertex lines preferably run upwards from distal to mesial. The boundary surface is thus preferably arranged obliquely in the ingot. This makes it possible to imitate the color and translucency of natural teeth, especially anterior teeth, particularly well.

In a further preferred embodiment, the second layer of the blank according to the invention has a continuous, i.e. linear, or discontinuous, i.e. non-linear, color gradient. This also allows the color and translucency gradient of natural teeth, in particular anterior teeth, to be imitated particularly well.

Particularly preferably, the zirconium oxide ceramic of the second layer contains yttrium and the color gradient, in particular a translucency gradient, is preferably formed by a gradient of the amount of yttrium, i.e. by a gradually changing amount of yttrium. Thus, the blank preferably exhibits a continuous, i.e. linear, or discontinuous, i.e. non-linear, gradient of the amount of yttrium. In particular, the content of yttrium increases from the boundary surface between the first and second layers towards the outer surface of the second layer opposite the boundary surface. This increase in the yttrium content may be continuous or incremental.

In one embodiment, the second layer of the blank according to the invention has at least two discrete layers that differ in their yttrium content.

Preferably, the second layer comprises an inner layer and an outer layer, the inner layer being adjacent to the boundary surface between the first and second layers and the outer layer being adjacent to the outer surface of the second layer opposite to the boundary surface. The inner layer serves in particular as a so-called dentin layer, i.e. to imitate the dentin area of natural teeth. The outer layer serves in particular as an incisal layer, i.e. to imitate an enamel layer of natural teeth.

In this embodiment, it is particularly preferred that
(a) the inner layer has an yttrium content of 2.0 to 6.0 mol %, in particular 3.0 to 5.0 mol %, and
(c) the outer layer has an yttrium content of 3.5 to 8.0 mol %, in particular 4.0 to 7.5 mol %,

wherein the yttrium content is defined as the proportion of the amount of substance of Y₂O₃ relative to the sum of the amounts of substance of Y₂O₃, ZrO₂ and HfO₂.

Optionally, there is a so-called intermediate layer between the inner layer and the outer layer. In the presence of an intermediate layer, it is particularly preferred that
(a) the inner layer has an yttrium content of 2.0 to 6.0 mol %, in particular 3.0 to 5.0 mol %,
(b) the intermediate layer has an yttrium content of 3.0 to 7.0 mol %, in particular 3.5 to 4.5 mol %, and
(c) the outer layer has an yttrium content of 3.5 to 8.0 mol %, in particular 4.0 to 7.5 mol %, wherein the yttrium content is defined as the proportion of the amount of substance of Y₂O₃ relative to the sum of the amounts of substance of Y₂O₃, ZrO₂ and HfO₂.

Furthermore, it is preferred that the inner layer, the outer layer and optionally the intermediate layer contain, in addition to Y₂O₃, oxides for adjusting the color, in particular oxides of Fe, Cr, Mn, Tb, Pr, Ce, Ni, Co, Nd, Dy, Eu, Er, V and/or Ti, and oxides for adjusting the sintering kinetics, in particular Mg, La, Al, Ce, Yb, Gd, Ga and/or In.

Preferably, the inner layer, the outer layer and optionally the intermediate layer further comprise, in addition to Y₂O₃, at least one oxide of Fe, Cr, Mn, Tb and Pr, and particularly preferably oxides of all of these elements, for adjusting the color. Preferably, the inner layer, the outer layer and optionally the intermediate layer further contain, in addition to Y₂O₃, at least one oxide of Mg, La, Y and Al, and particularly preferably oxides of all of these elements, for adjusting the sintering kinetics.

The second layer can preferably comprise at least one and in particular all of the following components in the amounts indicated:

| Component | Wt.-% |
|---|---|
| ZrO₂ + HfO₂ | 82.5-96.5, especially 84.5-95.0 |
| Y₂O₃ | 3.5-11.0, especially 5.0-9.0 |
| Fe₂O₃ | 0-0.15, especially 0.005-0.12 |
| Cr₂O₃ | 0-0.025, especially 0.0002-0.01 |
| Mn₂O₃ | 0-0.002, especially 0.00005-0.0012 |
| Pr₂O₃ | 0-0.02, especially 0.0001-0.015 |
| Tb₂O₃ | 0-0.02, especially 0.00005-0.015 |
| Er₂O₃ | 0-1.0, especially 0.01-0.65 |
| MgO | 0-0.05, especially 0-0.025 |
| Al₂O₃ | 0-0.25, especially 0-0.15 |
| La₂O₃ | 0-1.0, especially 0-0.55 |

Furthermore, the inner layer can preferably comprise at least one, and in particular all, of the following components in the amounts indicated:

| Component | Wt.-% |
|---|---|
| ZrO₂ + HfO₂ | 85.0-96.5, especially 86.5-95.5 |
| Y₂O₃ | 3.5-8.5, especially 4.5-7.0 |
| Fe₂O₃ | 0-0.15, especially 0.005-0.12 |
| Cr₂O₃ | 0-0.025, especially 0.0002-0.01 |
| Mn₂O₃ | 0-0.002, especially 0.00005-0.0012 |
| Pr₂O₃ | 0-0.02, especially 0.0001-0.015 |
| Tb₂O₃ | 0-0.02, especially 0.00005-0.015 |
| Er₂O₃ | 0-1.0, especially 0.01-0.65 |
| MgO | 0-0.025, especially 0-0.005 |
| Al₂O₃ | 0-0.25, especially 0-0.15 |
| La₂O₃ | 0-1.0, especially 0-0.55 |

Furthermore, the outer layer can preferably comprise at least one, and in particular all, of the following components in the amounts indicated:

| Component | Wt.-% |
|---|---|
| ZrO₂ + HfO₂ | 81.5-95.0, especially 82.5-93.5 |
| Y₂O₃ | 5.0-12.0, especially 6.5-11.0 |
| Fe₂O₃ | 0-0.1, especially 0.0035-0.085 |
| Cr₂O₃ | 0-0.025, especially 0.0002-0.01 |
| Mn₂O₃ | 0-0.002, especially 0.00005-0.0012 |
| Pr₂O₃ | 0-0.02, especially 0.0001-0.015 |
| Tb₂O₃ | 0-0.02, especially 0.00005-0.015 |
| Er₂O₃ | 0-0.6, especially 0.01-0.5 |
| MgO | 0-0.025, especially 0-0.005 |
| Al₂O₃ | 0-0.25, especially 0-0.15 |
| La₂O₃ | 0-0.55, especially 0-0.15 |

Furthermore, the intermediate layer can preferably comprise at least one and in particular all of the following components in the amounts indicated:

| Component | Wt.-% |
|---|---|
| ZrO₂ + HfO₂ | 83.0-96.0, especially 84.0-94.5 |
| Y₂O₃ | 4.0-11.0, especially 5.0-9.0 |
| Fe₂O₃ | 0-0.12, especially 0.005-0.1 |
| Cr₂O₃ | 0-0.025, especially 0.0002-0.01 |
| Mn₂O₃ | 0-0.002, especially 0.00005-0.0012 |
| Pr₂O₃ | 0-0.02, especially 0.0001-0.015 |
| Tb₂O₃ | 0-0.02, especially 0.00005-0.015 |
| Er₂O₃ | 0-1.0, especially 0.01-0.65 |
| MgO | 0-0.02, especially 0-0.005 |
| Al₂O₃ | 0-0.25, especially 0-0.15 |
| La₂O₃ | 0-1.0, especially 0-0.55 |

The zirconium oxide ceramic of the first layer comprises zirconium oxide that is preferably stabilized with erbium and/or yttrium. Preferably, the zirconium oxide ceramic of the first layer has an erbium content of 0.0 to 4.5 mol %, in particular 0.5 to 4.25 mol %, particularly preferably 1.5 to 3.5 mol %, the erbium content being defined as the proportion of the amount of substance of Er₂O₃ relative to the sum of the amounts of substance of Er₂O₃, ZrO₂ and HfO₂. Furthermore, the zirconium oxide ceramic of the first layer preferably has an yttrium content of 0.0 to 4.5 mol %, in particular 0.5 to 4.25 mol %, particularly preferably 0.75 to 2.0 mol %, the yttrium content being defined as a proportion of the amount of substance of Y₂O₃ based on the sum of the amounts of substance of Y₂O₃, ZrO₂ and HfO₂. Furthermore, it is preferred that the sum of the erbium and yttrium content is 1.5 to 6.0 mol %, in particular 2.0 to 4.5 mol %, more preferably 2.5 to 4.0 mol %. Since the first layer of the blank serves to form the gingival area of the dental restoration to be fabricated, a formation of a color gradient or material gradient of yttrium in the first layer is not advantageous. Thus, the yttrium content within the first layer is preferably substantially constant. Alternatively, the first layer may have a gradient to represent a fixed and mobile gingiva.

The zirconium oxide ceramic of the first layer preferably contains at least one, and in particular all, of the following components in the amounts indicated:

| Component | Wt.-% |
|---|---|
| ZrO₂ + HfO₂ | 86.0-97.0, preferably 90.0-94.0 |
| Er₂O₃ | 0.5-10.0, preferably 4.0-8.0 |
| Y₂O₃ | 0.5-8.0, preferably 1.0-4.0 |
| first coloring oxide | 0.0-0.5, preferably 0.0-0.25, |
| | particularly preferred 0.0-0.1 |
| second coloring oxide | 0.0-0.1, preferably 0.0-0.05, |
| | especially preferred 0.0-0.025, |

wherein the first coloring oxide is selected from the group consisting of Fe₂O₃, Tb₂O₃, Pr₂O₃ and V₂O₅ and in particular is capable of effecting an additional yellow coloring of the zirconium oxide ceramic, and wherein the second coloring oxide is selected from the group consisting of Mn₂O₃, Cr₂O₃ and CoO and in particular is capable of effecting an additional grey coloring of the zirconium oxide ceramic.

In one embodiment, it is preferred that the first layer of the blank comprises zirconium oxide ceramic or a mixture of zirconium oxide ceramic with one or more materials selected from the group consisting of alumina toughened zirconium oxide (ATZ), zirconium oxide toughened alumina (ZTA), spinels, pigments or mixtures thereof.

Alumina-reinforced zirconium oxide (ATZ) comprises 5 to 40 wt.-%, especially about 20 wt.-% Al₂O₃ and 60 to 95 wt.-%, especially about 80 wt.-%, ZrO₂. The Al₂O₃ portion may be doped with MgO, such as 50 wt.-ppm to 3 wt.-%. The ZrO₂ portion may be doped with Y₂O₃, such as 1 to 3 mol %.

Zirconium oxide-reinforced alumina (ZTA) comprises 60 to 95 wt.-%, in particular about 80 to 90 wt.-%, Al₂O₃ and 5 to 40 wt.-%, in particular about 10 to 20 wt.-%, ZrO₂. The Al₂O₃ portion may be doped with MgO, such as 50 wt.-ppm to 3 wt.-%. The ZrO₂ portion may be doped with Y₂O₃, such as 1 to 3 mol %.

In mixtures of zirconium oxide ceramics with ATZ and ZTA, discrete Al₂O₃ and ZrO₂ crystallites can be detected in the final structure by SEM. By contrast, in current zirconium oxide ceramics for dental applications, no Al₂O₃ crystallites can usually be detected in the microstructure by SEM. This is probably due to the smaller amount of Al₂O₂, usually about 0.05 to 0.1 wt.-%, and its homogeneous distribution. ATZ and ZTA composites can positively influence the mechanical properties of the blank, especially increasing both the biaxial strength and the fracture toughness. An increase in fracture toughness is achieved in particular when the ZrO₂ content of ATZ or ZTA is stabilized by 1.5 to 2.5 mol % Y₂O₃.

Spinels selected from the group consisting of MgAl₂O₄, SrAl₂O₄, La spinel, Y spinel and mixtures thereof, such as LaMg spinels, are particularly suitable as spinels. Spinels can positively influence the mechanical properties of the blank, in particular increase both the biaxial strength and the fracture toughness. An increase in fracture toughness is achieved in particular when the ZrO₂ portion of the zirconium oxide ceramic is stabilized by 1.5 to 2.5 mol % Y₂O₃. In the composite of zirconium oxide ceramic and spinel, the spinel usually grows in the form of rods, especially at high sintering temperatures, which has a positive effect on the fracture toughness of the composite.

While the second layer of the blank according to the invention is preferably tooth-colored and retains a tooth-colored shade even after heat treatment of the blank, the first layer can be whitish or pinkish and retain such a shade even after heat treatment. In the case of a first layer with a whitish shade, it is common to subsequently coat the gingival area after shaping and heat treatment in order to imitate the aesthetics of the gingival area in the finished dental restoration as closely as possible. Such a step can be omitted in the case of a first layer with a precolored first layer, so that in this embodiment a simpler production of e.g. dental prostheses is possible.

Particularly advantageous are blanks according to the invention in which the zirconium oxide ceramic of the first layer and the zirconium oxide ceramic of the second layer are pre-sintered. This improves the processability and precision during subsequent machining for the fabrication of dental restorations. In particular, due to the lower strength of zirconium oxide in the pre-sintered state, a simple, time-saving and milling tool-friendly shaping of the blanks is made possible. Preferably, the zirconium oxide ceramic of the first layer and the zirconium oxide ceramic of the second layer have a density of 1.8 to 4.4 g/cm³, in particular 2.5 to 4.0 g/cm³.

In the case of such pre-sintered blanks, it is necessary to subject the blanks to a sintering step, i.e. a heat treatment, for the fabrication of dental restorations, so that the desired mechanical properties, in particular a high strength and hardness, are achieved. During such a heat treatment, sintering shrinkage of the zirconium oxide ceramic occurs. It is thus necessary when using pre-sintered blanks that the undulating and radial geometry of the blanks according to the invention is enlarged compared to blanks based on materials without sintering shrinkage, for example plastic materials. Preferably, in a blank according to the invention in the pre-sintered state, the dimensions of the geometry are increased by a factor of from 1.200 to 1.250, in particular from 1.22 to 1.25. In the case of a green-state blank according to the invention, the dimensions of the geometry are increased by a factor of from 1.250 to 1.350, in particular about 1.275.

The blank according to the invention preferably has a biaxial fracture strength of 10 to 150 MPa, in particular 20 to 120 MPa, more preferably 25 to 80 MPa. The biaxial fracture strength was determined in accordance with ISO 8672 (2008) (piston-on-three-balls test).

Furthermore, the blank according to the invention preferably has a Vickers hardness Hv_{2.5} of 50 to 1000 MPa, in particular 300 to 850 MPa, more preferably 300 to 700 MPa. The Vickers hardness was measured according to ISO 14705: 2016 at a load of 2.5 kg.

Furthermore, it is preferred that the first layer and the second layer of the blank according to the invention are connected to each other by integral manufacture. This enables efficient monolithic production, i.e. a patient-specific total prosthesis can be produced from one blank in one milling process.

The shape of the blank according to the invention is preferably at least partially circular-arc-shaped. In particular, the blank has the shape of a disc, especially preferably the shape of a circular disc.

Furthermore, it is preferred that the circular-arc-shaped blank has a protrusion on the outer circumference, in particular an outwardly projecting clamping edge. This clamping edge serves as a holder in grinding and milling devices, such as common CAD/CAM devices. The protrusion can be made of the same material as the blank. In this case, the blank may first be provided with a larger circumference and the protrusion may be obtainable by machining, e.g. on a milling machine. Alternatively, the protrusion can be formed from a different material and can be formed, for example, by a plastic ring that is subsequently applied to the blank.

It is further preferred that the blank according to the invention has at least two markings, grooves and/or flattenings on the outer circumference, which are arranged asymmetrically and not rotationally symmetrically to each other, i.e. not diametrically opposite each other, and serve as rotation protection or anti-rotation protection. The position of the undulating geometry in relation to the disc must be known and fixed in all spatial directions so that the restoration can be placed in relation to the wave shape in the subsequent step for machining.

The zirconium oxide ceramic-based blank according to the invention is used in particular for fixed prosthetics. This means that the blank according to the invention is particularly suitable for the fabrication of fixed dental prostheses which are attached to implants or residual tooth parts, such as a tooth stump, by screwing or bonding. As a result, in the case of blanks according to the invention for the manufacture of a maxillary prosthesis, a fold and palatal plate, which are necessary for the adhesion of the prosthesis to the palate of the patient, can be omitted. This, in turn, means that the height of the first layer of the blank, which serves to mimic the gingival area, can be reduced. Furthermore, by eliminating the need for a palatal plate, it is not necessary to pink or otherwise color the entire first layer. Rather, when viewed from above the circular-arc-shaped base of the blank, an inner area can be omitted or left uncolored.

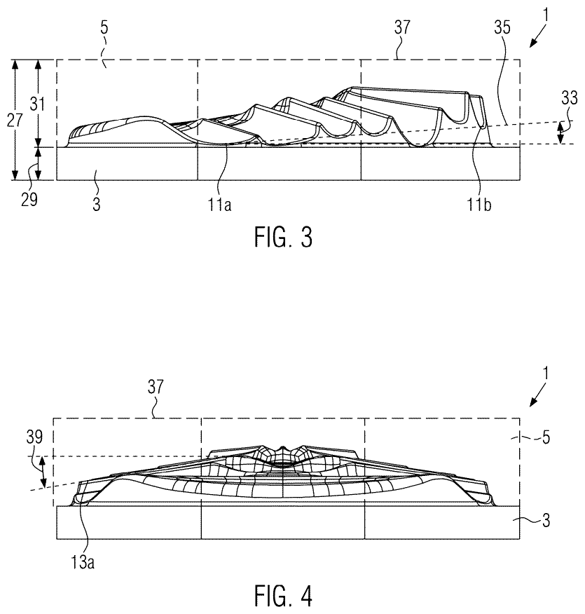

Consequently, in the case of blanks according to the invention, its height can be reduced so that it remains well below 30 mm, which is a critical limit for machining a blank in a commercially available dental milling machine. In a preferred embodiment, the height of the blank, i.e. the distance between the circular-arc-shaped top of the blank and the opposite circular-arc-shaped bottom of the blank, is not more than 30 mm, preferably 20 to 30 mm, more preferably 25 to 29 mm. Furthermore, the height of the first layer is preferably 5.0 to 9.0 mm, in particular 6.0 to 7.5 mm, particularly preferably about 7.0 mm, and/or the height of the second layer is preferably 15.0 to 25.0 mm, in particular 18.0 to 22.0 mm, particularly preferably about 20 mm. The height of the first layer is defined as the smallest distance between the outer surface of the first layer opposite the boundary surface and the deepest wave trough of the first layer in a side view of the outer surface of the preferably disc-shaped blank. The height of the second layer is defined as the smallest distance between the outer surface of the second layer opposite the boundary surface and the highest wave crest of the second layer in a side view of the outer surface of the preferably disc-shaped blank, as illustrated below as distance 31 in FIG. 3.

The ratio of the height of the first layer to the height of the second layer is preferably 1:1 to 1:5, in particular 1:2.5 to 1:3.5.

By combining the special wave geometry of the blank and the zirconium oxide material to be used according to the invention, further advantages can be achieved in addition to a reduction in the overall height of the blank. For example, it was surprisingly found that it is possible to reduce (i) the angle between the boundary surface between the first and second layer and the surface of the blank and/or (ii) the angle between the vertex line of the wave crests of the boundary surface and the surface of the blank compared to denture blanks known in the prior art.

In a preferred embodiment, which is particularly suitable for the preparation of a prosthesis in the upper jaw, the blank is characterized in that the angle between a fictitious straight line connecting the lowest point of the wave trough for the second molar to be fabricated with the lowest point of the wave trough for the central incisor to be fabricated, and the projection of this fictitious straight line onto the circular-arc-shaped base surface of the blank is 2.0° to 4.5°, preferably 2.0° to 4.0°, particularly preferably 2.5° to 3.5° and most preferably about 3.0°. By using such a small angle, the advantages of a color gradient, especially in the case of blanks with a color gradient in the second layer, can be better exploited, and the color and translucency gradient of anterior and posterior teeth of the prosthesis to be fabricated can be improved.

In a further preferred embodiment, the blank is characterized in that the angle between the circular-arc-shaped base surface of the blank and the vertex line of the wave crests of the undulating shaped boundary surface is 7° to 13°, preferably 9° to 11°, and particularly preferably about 10°.

In particular, in combination with a reduction of the overall height of the blank, the reduction of the angle of the boundary surface to the surface of the blank and/or the reduction of the angle of the vertex lines of the wave crests to the surface of the blank lead to an easier machinability of the blank in a milling machine, since in these embodiments less undercuts are necessary.

The invention also relates to a process for preparing the blanks according to the invention.

The process for preparing a blank according to the invention is characterized in that
(i) a first and a second green body are provided, wherein the first green body is provided for forming the first layer of the blank and the second green body is provided for forming the second layer of the blank, and wherein a first side of the first green body has the wave geometry of the first layer formed by wave trough and wave crest and a first side of the second green body has the corresponding inverse wave geometry;
(ii) the first and second green bodies are put on the top of each other so that the first side of the first green body and the first side of the second green body come into close contact with each other over their entire surface;
(iii) the first and second green bodies are pressed together; and
(iv) at least one heat treatment is carried out to convert the zirconium oxide into pre-sintered zirconium oxide.

Particularly preferably, a first green body and a second green body are provided separately in step (i) by filling the zirconium oxide ceramic starting materials for the first layer and the second layer each separately into a mold and subsequently pressing the masses each uniaxially and/or isostatically. The pressure during this pressing is preferably less than 100 MPa.

Subsequently, the desired shaping of the surface of the first and second green body can be achieved, preferably by milling.

Furthermore, it is preferred that the first and second green bodies are isostatically pressed together in step (iii), in particular at a pressure of more than 100 MPa, preferably at a pressure of 150 to 1000 MPa, preferably 150 to 500 MPa.

It is further preferred that in step (iv) the heat treatment takes place at a temperature of from 700 to 1200° C. and preferably at a temperature of from 800 to 1100° C. The duration of the heat treatment in step (iv) can preferably be from 5 to 600 min, in particular from 10 to 300 min, in a temperature range from 700 to 1200° C. and preferably from 800 to 1100° C.

Due to the described special properties of the blanks according to the invention, they are particularly suitable for the production of dental restorations, in particular dental prostheses. The invention therefore also relates to the use of the blanks according to the invention for the preparation of a dental restoration and in particular a dental prosthesis, wherein the dental prosthesis is preferably selected from the group consisting of maxillary full dentures, mandibular full dentures, maxillary partial dentures and mandibular partial dentures. In particular, said dental prostheses may advantageously be an implant restoration. In this case, the boundary surfaces of the dental prosthesis to the implants can be manufactured directly so that the dental prosthesis can be connected directly to the implant, e.g. by screw connection. Alternatively, the dental prosthesis can be connected to a base, e.g. a base made of titanium, e.g. by bonding, which is then connected to the implant by screwing.

In a further aspect, the invention also relates to a process for preparing a dental restoration comprising
(i-1) providing the shape of the dental restoration to a blank according to the invention by machining,
(i-2) carrying out at least one heat treatment to convert the zirconium oxide of the first and second layers into densely sintered zirconium oxide, and
(i-3) optionally finishing the surface of the dental restoration obtained.

The desirably shaped dental restorations, in particular dental prostheses, can be easily machined out of the blanks according to the invention.

The machining in step (i-1) is usually carried out by material-removing processes and in particular by milling and/or grinding. It is preferred that the machining is carried out with computer-controlled milling and/or grinding devices. Particularly preferably, the machining is carried out as part of a CAD/CAM process.

In step (i-2), the blank is subjected to a heat treatment to effect the formation of densely sintered zirconium oxide ceramic. The heat treatment takes place in particular at a temperature of 1050 to 1700° C. and preferably 1100 to 1600° C. The heat treatment is carried out in particular for a duration of 0 to 240 min, preferably 5 to 180 min, particularly preferably 30 to 120 min, the term “duration” referring to the holding time of the maximum temperature.

The dental restorations produced according to the invention are in particular dental prostheses and are particularly preferably selected from the group consisting of maxillary full dentures, mandibular full dentures, maxillary partial dentures and mandibular partial dentures.

In addition to excellent optical properties, the dental prostheses produced according to the invention are characterized by a particularly high strength. Preferably, the dental prosthesis has a biaxial fracture strength in the gingival area, i.e. in the first layer, according to ISO 6872 (2208) (piston-on-three-balls test) of more than 800 MPa, in particular more than 900 MPa and especially preferably more than 1000 MPa.

Furthermore, in the dentin area, i.e. in the inner layer of the second layer, the dental prosthesis preferably has a biaxial fracture strength according to ISO 6872 (2208) (piston-on-three-balls test) of more than 600 MPa, in particular more than 600 MPa and especially preferably more than 800 MPa.

In addition, the dental prosthesis in the incisal region, i.e. in the outer layer of the second layer, preferably has a biaxial fracture strength according to ISO 6872 (2208) (piston-on-three-balls test) of more than 300 MPa, in particular more than 400 MPa and particularly preferably more than 500 MPa.

In the optional step (i-3), the surface of the dental restoration can still be finished. In particular, it is possible to cover the gingival area of a dental prosthesis with a whitish gingival area, i.e. when using a blank with a whitish first layer, with layering, veneering, coloring or characterization materials in order to imitate the shade of natural oral mucosa particularly well. Alternatively or additionally, a surface polishing of the dental restoration can be carried out in step (i-3).

Further features and advantages will be apparent from the following description of embodiments of the present invention with reference to the drawings. It is shown in

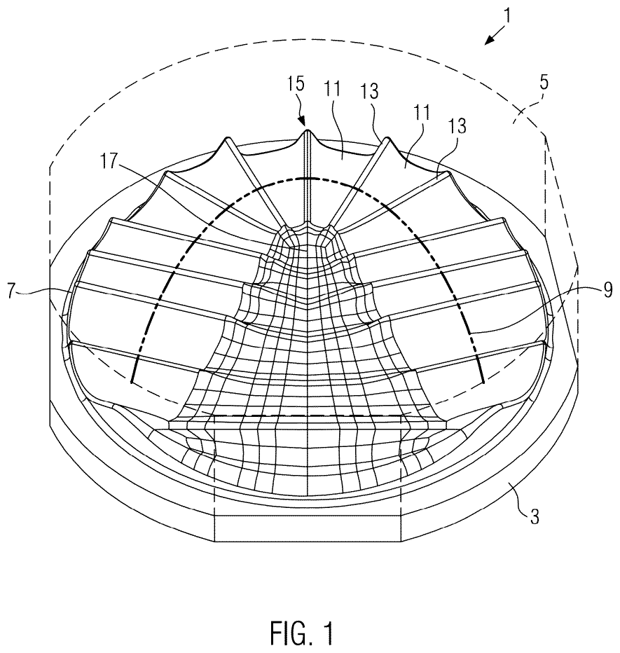

FIG. 1 a schematic perspective view of a blank according to the invention;

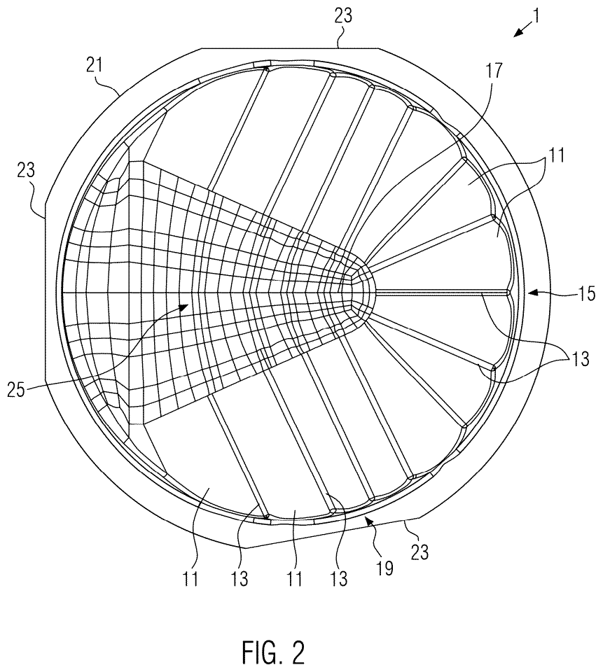

FIG. 2 a schematic top view of the first layer of the blank shown in FIG. 1;

FIG. 3 a schematic side view of a blank according to the invention; and

FIG. 4 another schematic side view of a blank according to the invention.

FIG. 1 shows a blank 1 according to the invention for a dental prosthesis for the upper jaw. The basic structure of the blank 1 is disc-shaped. It has a first layer 3 based on zirconium oxide ceramic and a second layer 5 based on zirconium oxide ceramic. The first layer 3 and the second layer 5 differ in color and form a boundary surface 7. In the illustration according to FIG. 1, the upper side of the first layer 3 and the lower side of the second layer 5 represent the boundary surface 7 of the blank. An exemplary schematic course of a dental arch, i.e. the course of the teeth of the denture to be made, is shown in FIG. 1 as line 9. In the course of the dental arch 9, the boundary surface 7 is formed in an undulating shape, with wave troughs 11 and wave crests 13 alternating. In the area 15 of the anterior teeth to be made, the wave crests 13 extend in a fan-shaped manner in the oral-vestibular direction. This means that the vertex lines of the wave crests 13 extend from a center point 17 of the dental arch 9 in a radial direction, i.e. outwards, towards the vestibular side, in a radial shape.

FIG. 2 shows a top view of the upper side of the first layer 3 of the blank 1 shown in FIG. 1. In the top view, the fan-shaped course of the wave crests 13 starting from the ray-center 17 can be seen in the front tooth region 15.

FIG. 2 also shows that the vertex lines of the wave troughs 11 and the wave crests 13 in the area 19 of the molars to be made run essentially parallel in the oral-buccal direction. As described above, this parallelization of the wave crests and wave troughs in the area of the molars ensures that a comparatively large occlusal surface is available in the finished dental prosthesis, even with small dental arches.

The blank according to FIG. 2 also has three flattenings 23 on its outer circumference, i.e. lateral surface 21, which are not arranged rotationally symmetrically to each other. The flattenings 23 serve as a marking in order to define the position of the undulating and radial geometry within the disc in relation to the disc itself, so that the disc can be placed correctly during the manufacture of the dental prosthesis. Thus, the flattenings 23 serve as rotation protection or anti-rotation protection during the subsequent milling process.

The inner area 25 of the first layer 3 represents the area of the prosthesis to be made that would come into contact with the patient's palate after insertion of the prosthesis, i.e. the so-called palatal plate. In the case of implant-or tooth stump-supported dentures, the area 25 can be omitted or can remain uncolored.

FIG. 3 shows a side view of the blank 1 shown in FIG. 1. The blank has an overall height 27, which in the specific embodiment of the blank shown in the figures is 26.80 mm. The height 29 of the first layer is 6.80 mm and the height 31 of the second layer is 20.00 mm. Compared to the blanks based on plastic materials known from EP 3 064 170 A1 and EP 3 597 143 A1, in which the height of the gingiva layer and the tooth layer is usually about 19.00 mm each, the overall height of the blank 1 has been significantly reduced and is below the critical limit of 30 mm, which allows easy machining in commercially available dental milling machines. In addition, the ratio of tooth layer to gingiva layer could be significantly increased, which allows better color and translucency gradients to be achieved.

Furthermore, blank 1 according to FIG. 3, is characterized in that the angle 33 between a fictitious straight line 35, which connects the deepest point of the wave trough 11a for the second molar to be made with the deepest point of the wave trough 11b for the central incisor to be made, and the projection of this fictitious straight line 35 onto a base surface of the blank, in this case the upper surface 37 of the blank, is 3° and is thus smaller than the corresponding angle in the case of blanks based on plastic materials known from EP 3 064 170 A1 and EP 3 597 143 A1, which is usually 5° there. By reducing the angle 33, the color and translucency of the anterior and posterior teeth of the prosthesis to be fabricated can be further improved.

FIG. 4 shows a further side view of the blank 1 shown in FIG. 1. The angle 39 between the surface 37 and the vertex line of the wave crests 13a is 10° in the embodiment shown and is thus significantly smaller than the corresponding angle in blanks based on plastic materials known from EP 3 064 170 A1 and EP 3 597 143 A1, where it is usually 15°. The reduction of the angle 39, like the reduction of the angle 33, leads to simpler machining of the blank in a milling machine, as fewer undercuts are necessary in these embodiments.

1. A blank for dental prostheses comprising
a first layer based on zirconium oxide ceramic and
a second layer based on zirconium oxide ceramic,
the first layer and the second layer differing in color and forming a boundary surface,
wherein the boundary surface is formed, in the course of a dental arch, in an undulating shape with alternating wave troughs and wave crests, and vertex lines of the wave crests, viewed in plan view of the boundary surface, extend radially in a mesial-distal direction.

2. The blank according to claim 1,
wherein the second layer has a continuous or discontinuous color gradient.

3. The blank according to claim 2,
wherein the zirconium oxide ceramic of the second layer comprises yttrium and the color gradient is formed by a gradient of the content of yttrium.

4. The blank according to claim 3,
wherein the content of yttrium in the second layer increases from the boundary surface to an outer surface of the second layer opposite the boundary surface.

5. The blank according to claim 3,
wherein the second layer comprises an inner layer and an outer layer, the inner layer being adjacent the boundary surface between the first and second layers and the outer layer being adjacent the outer surface of the second layer opposite the boundary surface.

6. The blank according to claim 5, wherein
(a) the inner layer has an yttrium content of from 2.0 to 6.0 mol %, and
(c) the outer layer has an yttrium content of from 3.5 to 8.0 mol %,
wherein the yttrium content is defined as the proportion of the amount of substance of Y₂O₃ relative to the sum of the amounts of substance of Y₂O₃, ZrO₂ and HfO₂.

7. The blank according to claim 1,
wherein the first layer is made of zirconium oxide ceramic or a mixture of zirconium oxide ceramic with one or more materials selected from the group consisting of alumina reinforced zirconium oxide, zirconium oxide reinforced alumina, spinels, pigments or mixtures thereof.

8. The blank according to claim 1,
wherein the zirconium oxide ceramic of the first layer comprises erbium and/or yttrium, the first layer having an erbium content of 0.0 to 4.5 mol %, wherein the erbium content is defined as the proportion of the amount of substance of Er₂O₃ relative to the sum of the amounts of substance of Er₂O₃, ZrO₂ and HfO₂, and/or an yttrium content of 0.0 to 4.5 mol %, wherein the yttrium content is defined as a proportion of the amount of substance of Y₂O₃ relative to the sum of the amounts of substance of Y₂O₃, ZrO₂ and HfO₂, the sum of the erbium and yttrium content being 1.5 to 6.0 mol %.

9. The blank according to claim 1,
wherein the first layer is whitish or pinkish and the second layer is tooth-colored.

10. The blank according to claim 1,
wherein the zirconium oxide ceramic of the first layer and the zirconium oxide ceramic of the second layer are pre-sintered,
wherein the zirconium oxide ceramic of the first layer and the zirconium oxide ceramic of the second layer, independently of each other, preferably have a density of 1.8 to 4.4 g/cm³.

11. The blank according to claim 1,
wherein the first layer and the second layer are connected to each other by integral manufacture.

12. The blank according to claim 1,
which is at least partially circular-arc-shaped and has the shape of a disc.

13. The blank according to claim 12,
wherein a protrusion having an outwardly projecting clamping edge is formed on an outer circumference of the blank.

14. The blank according to claim 1,
wherein the boundary surface, viewed in plan view of the boundary surface, in a region of anterior teeth to be produced has, in an oral-vestibular direction, vertex lines of wave crests in a fan shape.

15. The blank according to claim 1,
wherein the boundary surface, viewed in plan view of the boundary surface, in a region of molars to be produced has, in an oral-buccal direction, radiating vertex lines of the wave troughs or vertex lines of the wave troughs which are substantially parallel to one another.

16. The blank according to claim 12,
wherein an angle between a fictitious straight line connecting a lowest point of the wave trough for a second molar to be produced with a lowest point of the wave trough for a central incisor to be produced, and the projection of said fictitious straight line onto a base surface of the circular-arc-shaped blank is 2.0° to 4.5°.

17. The blank according to claim 12,
wherein a height of the blank is not more than 30 mm, wherein (i) a height of the first layer is 5.0 to 9.0 mm, and/or (ii) a height of the second layer is 15.0 to 25.0 mm.

18. The blank according to claim 12,
wherein an angle between a base surface of the circular-arc-shaped blank and a vertex line of the wave crests of the undulating boundary surface is 7° to 13°.

19. A process for preparing a blank according to claim 1, comprising
(i) providing a first and a second green body, wherein the first green body is provided for forming the first layer of the blank and the second green body is provided for forming the second layer of the blank, and wherein a first side of the first green body has a wave geometry of the first layer formed by a wave trough and a wave crest and a first side of the second green body has a corresponding inverse wave geometry;
(ii) putting the first and second green bodies on the top of each other so that the first side of the first green body and the first side of the second green body come into close contact with each other over their entire surface;
(iii) pressing the first and second green bodies together; and
(iv) carrying out at least one heat treatment to convert the zirconium oxide into pre-sintered zirconium oxide.

20. The process according to claim 19,
wherein in step (iii) the first and second green bodies are isostatically compressed together at a pressure of more than 100 MPa.

21. The process according to claim 19,
wherein in step (iv) the heat treatment takes place at a temperature of from 700 to 1200° C.

22. (canceled)

23. A process for preparing a dental restoration, comprising
(i-1) shaping the dental restoration from the blank according to claim 1 by machining,
(i-2) carrying out at least one heat treatment to convert the zirconium oxide of the first and second layers into densely sintered zirconium oxide, and
(i-3) optionally finishing the surface of the dental restoration obtained.

24. (canceled)

25. (canceled)
